(19) United States
(12) Patent Application Publication
(10) Pub. No.: US 2016/0220520 A1
(43) Pub. Date: Aug. 4, 2016

(54) APPARATUS, SYSTEM AND METHOD FOR THE TREATMENT OF ATHEROSCLEROSIS, HEART DISEASE AND STROKE

(71) Applicants: Steve Andre Beaudin, Ottawa (CA); Catherine Shuqi Xu Beaudin, Ottawa (CA)

(72) Inventors: Steve Andre Beaudin, Ottawa (CA); Catherine Shuqi Xu Beaudin, Ottawa (CA)

(73) Assignees: Steve Beaudin, Ottawa (CA); Catherine Beaudin

(21) Appl. No.: 14/608,197

(22) Filed: Jan. 29, 2015

(51) Int. Cl. A61K 31/202; A61M 1/34; A61K 31/045; A61M 5/36; A61K 31/201; A61K 31/20

(52) U.S. Cl. CPC A61K 31/202; A61K 31/201; A61K 31/20; A61K 31/045; A61M 5/36; A61M 1/342; A61M 2205/75; A61M 2202/0468

(57) ABSTRACT

Biocompatible solvents, systems, apparatus and methods are provided for treating atherosclerosis, heart disease and stroke by dissolving and reducing arterial plaque within a patient's cardiovascular system. A biocompatible solvent is described based on desired solubility parameters and molecules of free fatty acids and other compositions which are known to be safe for humans have been identified which meet the required criteria. Furthermore, the physiological implications of injecting the biocompatible solvents into a patient intravenously are considered and the interaction with blood proteins discussed and considered. Finally, a system and apparatus to inject the biocompatible solvents into a patient as well as to filter and remove the solvent from the patient's cardiovascular system post injection are described.

### CROSS REFERENCE TO RELATED APPLICATIONS

This application claims priority from U.S. Provisional Application No. 61/932,781 filed Jan. 29, 2014, entitled “Apparatus, System and Method for the Treatment of Atherosclerosis and other Conditions Related to Fat or Lipid Accumulation in the Body” as well as U.S. Provisional Application No. 61/975,006 filed Apr. 4, 2014, also entitled “Apparatus, System and Method for the Treatment of Atherosclerosis and other Conditions Related to Fat or Lipid Accumulation in the Body” which are both incorporated herein by reference.

### TECHNICAL FIELDS

The present invention generally relates to a system, method and apparatus for treating atherosclerosis by progressively reducing the build up of cholesterol and plaque from the walls of a patient's arteries. The invention will find application in treating and preventing heart disease and stroke. The invention may also find applications for weight control by helping remove fat from an obese individual. The invention more specifically relates to a method, system and apparatus for dissolving arterial plaque in-vivo and processing blood, and removing dissolved cholesterol, cholesterol-esters or other fats or lipids from a persons cardiovascular system to provide an overall reduction of these compounds in a patient's metabolism and more specifically the arteries The invention relates to both the use of biocompatible solvents which can be administered intravenously and a medical device which can be used to administer the biocompatible solvents as well as remove them from the patients circulatory system during and following the course of treatment. The biocompatible solvents can be used stand alone, without the medical device, if they are used in small repeated doses over a protracted treatment period. Use of the medical device in conjunction with the biocompatible solvents allows a more aggressive treatment to be implemented and larger quantities of the biocompatible solvent to be used in a single treatment.

### BACKGROUND

Cholesterol and fats are essential for life. However, the link between heart disease and high levels of cholesterol and fats is well established. High levels in blood circulation, depending on how it is transported within lipoproteins, are strongly associated with the progression of atherosclerosis. For a person of about 68 kg, typical total body cholesterol synthesis is about 1 g per day, and total body content is about 35 g. Typical daily additional dietary intake in the United States is 200-300 mg. The body compensates for cholesterol intake by reducing the amount synthesized.

Cholesterol is only slightly soluble in water, with a solubility of 0.000095 grams/litre at 30° C. Since cholesterol can dissolve and travel in the water-based bloodstream at exceedingly small concentrations, it is transported in the circulatory system within lipoproteins. Lipoproteins are complex discoidal particles which have an exterior composed of amphiphilic proteins and lipids whose outward-facing surfaces are water-soluble and inward-facing surfaces are lipid-soluble. Lipoproteins carry triglycerides and cholesterol esters are carried internally. Phospholipids and cholesterol, being amphipathic, are transported in the surface monolayer of the lipoprotein particle.

“In addition to providing a soluble means for transporting cholesterol through the blood, lipoproteins have cell-targeting signals that direct the lipids they carry to certain tissues. For this reason, there are several types of lipoproteins within blood called, in order of increasing density, chylomicrons, very-low-density lipoprotein (VLDL), intermediate-density lipoprotein (IDL), low-density lipoprotein (LDL), and high-density lipoprotein (HDL). The more lipid and less protein a lipoprotein has, the less dense it is. The cholesterol within all the various lipoproteins is identical, although some cholesterol is carried as the “free” alcohol and some is carried as fatty acyl esters referred to as cholesterol esters. However, the different lipoproteins contain apolipoproteins, which serve as ligands for specific receptors on cell membranes. In this way, the lipoprotein particles are molecular addresses that determine the start- and endpoints for cholesterol transport.”¹

In developed countries, the availability of clean water, adequate food and advanced health care services have resulted in significantly increased life spans. However, heart disease and more specifically atherosclerosis and atheroma processes have become an increasingly important problem and burden for society. In North America and Europe, atheroma continues to be the number one underlying basis for disability and death, despite substantial efforts to educate the public on risk factors and research into pharmaceutical treatments. Thus, increasing efforts towards better understanding, treating and preventing the problem are continuing to evolve.

### DISCLOSURE OF INVENTION

The current invention is aimed at removing undesirable fatty compounds such as arterial plaque, cholesterol, cholesterol esters and other fatty compounds from the cardiovascular system or body of a patient using biocompatible solvents either stand alone or in conjunction with a medical device to establish a physical transport mechanism. The undesirable fatty compounds can be any one of arterial plaque, cholesterol, cholesterol esters, fat, or any other fatty compounds which we desire to remove from a patient. The invention comprises of injecting a biocompatible solvent means intravenously into a vein or artery to allow the biocompatible solvent means to flow through a patient's cardiovascular system such that it dissolves arterial plaque, or other undesirable fatty compounds from the arterial walls of a patient. A medical device is also disclosed which is used in conjunction with the biocompatible solvents to administer the biocompatible solvent into the patient as well as filter the patients blood and remove the biocompatible solvent from the patients cardiovascular system. The biocompatible solvent means can be used without the medical device if smaller doses are used in repeated treatments and the biocompatible means is allowed to be metabolized by the patient. Use of the biocompatible solvent means in conjunction with the medical device allows more aggressive treatments since the biocompatible solvent means can be injected intravenously, and then subsequently filtered and removed by the medical device such that it need not be completely metabolized by the patient. This allows greater quantities of the biocompatible solvent means to be circulated through a patient's cardiovascular system and thereby allows more arterial plaque to be dissolved in a single treatment. The medical device establishes a physical transport mechanism by using an extracorporeal blood circuit to remove the said undesirable fatty compounds such as cholesterol, cholesterol esters or fatty compounds which are being transported in a patient's blood, or dissolved within the droplets of the biocompatible solvent, by trapping them in a filtration and precipitation unit. The filtered blood which has a lower concentration of these dissolved materials can be re-injected into the patient along with additional amount of the biocompatible solvent, causing more arterial plaque, cholesterol or fatty compounds to dissolve from the patients arterial walls or body, into the blood stream, which can then be further processed and removed from the blood stream. As some of the biocompatible solvent means is removed from the patient's circulation, additional amount of the biocompatible solvent means can be injected to keep the concentration of the biocompatible solvent means at a desired concentration.

The biocompatible solvent means can be any compound which can be injected into a patient intravenously and can be shown to dissolve arterial plaque, cholesterol, cholesterol esters and fatty compounds within the patient when used at concentrations which are considered safe for the patient. The biocompatible solvent means can be water soluble, water insoluble or amphipathic. A biocompatible solvent means which is water insoluble is particularly effective since the biocompatible solvent will not mix with the water based blood and as small droplets of the water insoluble solvent travel through the patients cardiovascular system, they bump and make contact with arterial plaque on the walls of arteries and cause small amounts of arterial plaque to dissolve into the droplet of the water insoluble solvent. As the droplet is carried away from the plaque by the flowing blood, a small quantity of the cholesterol or fatty material which dissolved into the droplet, is carried away with it, thereby decreasing the size and quantity of the plaque on the patients arteries. The patient's blood can then be circulated through an extracorporeal medical device where the droplets of the water insoluble solvent are removed from the blood stream by a filtration and precipitation means after which the blood is re-injected into the patient. As some of the water insoluble solvent is removed from the patient and trapped in the filtration and precipitation means, additional amounts of the biocompatible solvent means are injected into the patient's blood stream to keep the concentration at a desired value. The closed loop circuit and the mass transfer process can be maintained for many hours per treatment until a desired amount of arterial plaque, cholesterol, cholesterol esters or other fatty compounds have been removed from the patient.

The biocompatible solvent means can preferably comprise of any compound or mixture of compounds which are reasonably insoluble in water or blood, and which are known to have the ability to dissolve arterial plaque, cholesterol, cholesterol esters or other fatty compounds which are desirable to remove from a patient's body. To effectively dissolve arterial plaque in-vivo it is very important that the biocompatible solvent be sufficiently insoluble in blood to allow a small discrete droplet to travel through the blood stream, from the injection point, to the plaque which is being treated. If the biocompatible solvent dissolves within the blood, such as an alcohol, the solubility parameters of the resultant mixture will be very nearly identical to that of blood and will not dissolve cholesterol or arterial plaque. Conversely, if a discrete little droplet of the biocompatible solvent makes contact with the plaque, then the cholesterol molecule in the plaque will be surrounded by multiple molecules of the biocompatible solvent, which have similar solubility parameters as the cholesterol, and the cholesterol will be drawn away from the plaque and into the droplet. Some of the water insoluble solvents which have been tested and are showing very promising results are Alpha-linolenic acid also known as Omega 3, Linoleic acid also known as Omega 6, oleic acid also known as Omega 9, Elcosapentaenoic acid also known as EPA Omega3, Docosahexaenoic Acid also known as DHA Omega 3, capric acid (a medium chain saturated fatty acid common in plant and animal milk), 1,4-dioxane, and some brominated vegetable oils such as 1-Bromonaphthalene. Salts, Esters, an organo-metallic compound could also be used as the main component or an addition to the biocompatible solvent means. Although 1,4-dioxane and 1-Bromonaphthalene are toxic in reasonable quantities, they are included in this list since their solubility parameters are extremely well matched to those of cholesterol. An organic salt or organometallic compound could also potentially be used. Essentially any organic molecule which can be administered intravenously with acceptable side effects, is sufficiently insoluble in blood to travel as a discrete droplet and which is capable of dissolving cholesterol or the target material, could be used. A good example are bile acids, or similar molecules, which have both a hydrophobic and hydrophilic end and form micelles within the water based blood. These micelles draw hydrophobic substances such as fats and cholesterol within their core, and the hydrophilic tail of the molecules point outwards toward the blood to allow them to travel easily within the water based blood. Furthermore, compounds which are water soluble such as an alcohol or detergent, can be mixed with the water insoluble compound to arrive at a solution which is nevertheless insoluble in blood but has solubility parameters which are a better match to those of cholesterol. Furthermore, a combination of several compounds can be used to improve the rate of mass transfer and to reduce potential toxic side effects that a large quantity of a single compound might cause. The ideal water insoluble solvent must have solubility parameters which are a good match to the fatty compounds being targeted, be non-toxic or have acceptably low toxicity to the patient in quantities which will be administered and sufficiently water or blood insoluble so that the biocompatible solvent means can travel through the blood stream as tiny droplets. Given that the solubility parameters of a solution is equal to the weighted average of the Hansen solubility parameters of the individual components, it is possible to mix 2 or more compounds together to arrive at a solvent which is better suited to dissolving cholesterol than any of the components alone.

If the biocompatible solvent means is going to be used in conjunction with the medical device which includes and filtration and precipitation means, the ideal biocompatible solvent should have physical properties which allow it to be easily separated from the blood stream once it is in the medical device. Three properties which can be leverage to effect separation of the solvent from the blood are the density, the freezing temperature as well as the hydrophobic characteristics. For example, most of the water insoluble solvent tested so far have a density which is lower than blood and as such can be separated from the patient's blood stream by causing them to float to the top of a separation column or centrifugal filter and taking blood which is mostly free of the biocompatible solvent means from the bottom of a separation column. The freezing temperature can also be used to advantage by cooling the blood below the freezing temperature of the said water insoluble solvent. For example, blood can be cooled to nearly 0° C. in the medical device without damaging the blood cells, while Oleic acid has a freezing temperature of 6° C. Causing the droplets to solidify make it easy to separate them using a fine mechanical filter. Cholesterol and free fatty compounds may also solidify on the internal walls of the heat exchanger which is used to cool the blood or alternatively may form liquid or solid particulates in the blood. When using a water insoluble solvent however cooling the blood in the extracorporeal unit is not necessary since we can use two other properties such as density or their hydrophobic characteristics to separate them from the blood. The mass transport mechanism and the water insoluble solvent can be established without the need to cool the blood in the extracorporeal device although cooling can be used advantageously under certain circumstances. Better performance may be obtained by cooling but it may not be required. Solid particulates are removed using a specially designed filter. Liquid particulates can also be removed by designing the filter to cause these substances to float and accumulate at the top while the blood will flow towards the bottom and be separated. If the blood was cooled in the medical device then the device can optionally re-heat the filtered blood back to a temperature near 37° C. before re-injecting the processed blood into the patient, to prevent from cooling the patient and potentially causing the onset of hypothermia. By systematically removing the said water insoluble solvent which is carrying the dissolved cholesterol and fatty compounds from a person's blood stream, and re-injecting processed blood with a very low concentration of dissolved cholesterol and additional amounts of the said water insoluble solvent into the patient, more cholesterol and fatty materials are caused to dissolve off a persons arterial walls or from their body and into the droplets of the biocompatible solvent means which are travelling through the blood stream which can then further be filtered and removed from circulation by the filtration and precipitation means within the medical device. This extra-corporeal loop establishes a very effective method of extracting cholesterol or other fatty compounds from an individual's cardiovascular system or body. The system and method can be used to treat heart disease, stroke, or atherosclerosis. Given that many of the biocompatible solvent means which have been named above are actually contained in common foods and oils, it is important to review blood chemistry as well as the method in which lipids are absorbed and carried within the body to properly understand the invention being disclosed herein. For example, Alpha-linolenic acid also known as Omega 3, Linoleic acid also known as Omega 6, oleic acid also known as Omega 9, Elcosapentaenoic acid also known as EPA Omega3, Docosahexaenoic Acid also known as DHA Omega 3 are contained in common foods and are very safe for human consumption, and many of these molecules have even been shown to be beneficial in preventing heart disease. These molecules are generally known as fatty acids, and more specifically long chain fatty acids since they all contain between 12 and 22 carbon. Although we consume these fatty acids in everyday food, similarly to cholesterol, they are very water insoluble and as such cannot travel within the water based blood stream as free fatty acids but must be carried within lipoproteins.

Dietary fatty-acids are absorbed differently depending on the size of the molecule. As food is digested within the intestine and fatty acids are released from the food, the fatty acids will dissolve through the lining of the intestine. Short and medium chain fatty acids (2-10 carbon atoms) which are moderately water soluble are absorbed directly into the blood of the stomach lining and travel through the portal vein towards the liver where they are processed. The portal vein is not a true vein since it does not carry blood back towards the heart but is a dedicated blood vessel which carries nutrient rich blood directly from the gastrointestinal (make one word, checked on the internet) lining back to the liver. Long Chain fatty acids (12-22 carbon atoms) and Very Long Chain fatty acids (greater than 22 carbon atoms) are not soluble in the blood and therefore cannot be transported directly in the portal vein. The body has developed a fairly complex process to absorb and carry these long chain fatty acids. Given that they cannot be carried in the blood due to their insolubility, they are instead assembled into triglycerides and combined with protein to create a chylomicron directly within the villi of the intestinal wall. The chylomicron is a lipoprotein which originates directly within the lining of the intestine for the purpose of carrying the long chain fatty acids. Similarly, very low density lipoproteins (VLDL) are also produced in the intestinal wall and used to transport very long chain fatty acids. The chylomicrons are then transported from the intestine through the lymphatic system and enter the cardiovascular system through the thoracic duct which drains into the subclavian vein. From there, the chylomicrons travel through the cardiovascular system and can be absorbed in various tissue such as adipose tissue, or are eventually processed by the liver. Long and very long chain fatty acids which are stored in the liver, can be combined into Very Low Density Lipoproteins (VLDL), or Low Density Lipoproteins (LDL) and carried back out from the liver to other parts of the body such as adipose tissue. Some fatty acids may be released into the blood stream by adipose cells at which point albumin, a blood protein, will collect the fatty acids and carry them back to the liver. Albumin is a blood protein who's surface is mostly water soluble and as such is dissolved within the blood, but has a few hydro-phobic receptor sites to which water insoluble fatty acids can bind

It is interesting to note that although the human body needs and uses sizable quantities of long chain fatty acids and very long chain fatty acids, given they are not water soluble they are almost always carried about by lipoproteins. As such, droplets of fatty acid will never come into direct contact with atherosclerotic plaque in an individual's arteries. In the invention being disclosed here, fatty acids are being used as a component in the biocompatible solvent means and these compounds are being injected intravenously directly into a patient's blood stream such that they make contact with arterial plaque. Each droplet dissolves a small quantity of cholesterol or other fatty compound from the plaque and thereby results in meaningful reduction of the plaque.

If the biocompatible solvent means is used without the medical device, the solvent can be directly injected into a vein or artery. If a patient is known to have an artery which needs to be treated, a more focused treatment can be administered by inserting a catheter or small needle upstream of the artery which needs to be treated, and injecting the biocompatible solvent means slightly upstream of the plaque being treated. Droplets of the biocompatible solvent means will then make contact with the plaque as they flow through the artery and dissolve small quantities of cholesterol and other fatty materials from the plaque. As the droplets travel downstream, they carry away the dissolved cholesterol from the area being treated. The droplet will eventually be pushed through the capillaries and make its ways towards the venous system all the while the albumin will be binding with molecules of the fatty acid within the droplet and allow it to be metabolized. The minute quantity of the cholesterol which had been dissolved within the droplet will also be absorbed by the albumin or other lipoproteins and be carried back to the liver or reabsorbed into other cells. If sufficiently large quantities of the biocompatible solvent means are used, all the receptor sites of the available albumin may be used up at which point the droplets will continue to travel through the cardiovascular system as discrete little droplets. The medical device along with the filtration means can be used to separate the droplets of the biocompatible solvent from the blood flow. If excessive amounts of the biocompatible solvent are administered during treatment and/or the medical device with a filtration means is not being used, it may be beneficial to administer an injection of human albumin to the patient.

The composition of blood should also be discussed and understood in order to properly consider the operation of the filtration and precipitation means within the medical device.

Blood is typically categorized into 2 main parts, 55% blood plasma and 45% blood cells. Blood plasma is composed primarily of water (92% by volume) along with 6-8% of dissolved blood proteins (albumin, VLDL, LDL, HDL, etc), iron, glucose, clotting factors, electrolytes (Na⁺, Ca²⁺, Mg²⁺, HCO₃⁻, Cl⁻, etc.), hormones, and carbon dioxide. Blood cells are actual formed entities which include red blood cells, white blood cells and platelets. Blood cells are not dissolved in the blood but rather in suspension. This contrasts with the blood proteins and minerals which are actually soluble in the blood plasma and are therefore dissolved. For the purpose of this invention it is important to note that although some of the lipoproteins such as the chylomicrons, VLDL and LDL have very low densities, they are dissolved within the blood and as such will not be separated from the blood based on their densities. Blood cells are discrete objects which are in suspension within the blood and could potentially be separated from blood based on their higher densities, however they have a higher density that water and are denser than the fatty acids being proposed as the biocompatible solvent means. In general, the average density of whole blood is approximately 1.06 g/cm³, while the average density for blood cells is approximately 1.125 g/cm³. Blood plasma has a slightly lower density of approximately 1.025 g/cm³. In contrast, the density of some of the fatty acids which are showing best results such as oleic acid is only 0.895 g/cm³ and α-Linolenic acid is 0.914 g/cm³ and as such droplets of such compounds could be easily separated from the blood in the filtration system based on density alone.

Given that arterial plaque, cholesterol, cholesterol esters and other fatty compounds have a very low solubility in water, or blood, the water insoluble biocompatible solvent means is required to dissolve the fatty compounds we are targeting and allow it to be transported through the cardiovascular system and into the device, or be metabolized. Solubility parameters can be used to predict or estimate how well a potential water insoluble solvent will dissolve the target material. Solubility theory is a very large and complex field but one of the best and most widely accepted theories was developed by Charles Hansen in 1969. The Hansen solubility theory defines three parameters which are known as the HSP values for a compound. The HSP values quantify intermolecular forces by considering three distinct Van der Wall forces for a compound, specifically: London Dispersion Forces δ_{d}, Keesom Polarity Forces δₚ (between permanent dipoles), and Hydrogen Bonding δₕ. Molecules which have strong permanent dipoles (polar molecules) will have a fairly large δₚ parameter. Hydrogen bonding is a very strong form of polar bonding which occurs between a hydrogen atom and either a nitrogen, oxygen or fluorine atom with very high electron affinity. Water, H₂O, the main component of blood, has a very strong parameter for polar and hydrogen bonding given it is a polar molecule with hydrogen and oxygen atoms. Most undesirable fatty compounds tend to have very small coefficients of δₚ and δₚ and are therefore insoluble in water or blood.

The extend to which two compounds will dissolve each other can be quantified using the equations developed by Hansen:

(Ra)²=4(δ_{d2}−δ_{d1})²+(δₚ₂−δₚ₁)²+(δₕ₂−δₕ₁)² Eq. (1))

RED=Rₐ/Rₒ Eq. (2)

Equation 1 calculates the relative magnitude difference of the three Hansen parameters of the two compounds to arrive at Ra. The smaller the value of Ra, the better the two compounds will dissolve each other. Equation 2 allows one to calculate the Relative Energy Difference (RED) of the solvent and the material we are trying to dissolve, by dividing Ra by Ro. Ra was calculated in Equation 2 and measures the magnitude of the difference of the HSP values of the two compounds. Rₒ is the interaction radius over which the compound we desire to dissolve has been shown to interact with potential solvents. For an RED>>1 the compounds will not dissolve. For an RED˜1, there will be some dissolution. For an RED<1 there will be a considerable amount of dissolution.

Tables 1 provides the Hansen solubility parameters for a few select solvents and water. As can be seen, water which is the main component of blood has very strong polar bonding and hydrogen bonding components, and the RED value with both fat and cholesterol is considerably larger than 1, which explains why cholesterol and fat are insoluble in water. We then provide the HSP parameters for 3 alcohols which are known to be safe for human consumption is small quantities, namely Ethanol, Propanol and Butanol. The RED values for cholesterol with each of Ethanol, Propanol and Butanol is 1.32, 1.10 and 0.944 respectively. The mass fraction of cholesterol which will dissolve in a pure solution of each of these alcohols at 37° C. is 3.3%, 11% and 11.8% respectively². As can be seen, smaller RED values leads to greater solubility's. For cholesterol in water the RED value is 2.9 and the mass fraction of cholesterol which will dissolve in pure water is 0.0000095% which is very negligible. One of the complications of using a water soluble solvent such as the alcohols mentioned above, is that they will mix with water. If we mix two solvents together, the results HSP parameters of the solution is the weighted average of the HSP of the individual components. Since the safe concentration of alcohol which can safely be in a patient's blood is below 1%, the HSP of the blood/alcohol solution will be very similar to that of the blood without the alcohol. Conversely, using a solvent which is “water insoluble” allows little droplets of the solvent to travel through the blood stream which are going to have HSP parameters which are very well matched to the cholesterol or undesirable fatty compound we want to remove from the patient, and will dissolve the target material on contact. The droplets will then carry the dissolve undesirable fatty compounds through the blood stream and will eventually be trapped within the precipitation/filtration unit of the medical device, or be metabolized. As can be seen, Oleic acid has HSP parameters which are well matched to both cholesterol and fat. Oleic acid is the main component in olive oil and is very safe for human consumption. Relatively large quantities of Oleic acid could be circulating through a patient's blood stream without ill effect. Furthermore, Oleic acid has a density which is considerable less than blood (0.895 vs 1.06 g/ml) and could easily be separated from the blood stream based on this physical parameter. Also, Oleic acid will freeze at about 13° C., and as such could be caused to solidify in the medical device by chilling the blood down to near 0° C. temperatures. We have not yet determined the HSP parameters of Alpha-linolenic acid, Linoleic acid, Elcosapentaenoic acid also known as EPA Omega3 and Docosahexaenoic Acid also known as DHA Omega 3 since these are not commonly used in the chemical processing industry. However, we have shown through simple solubility experiments that they are indeed able to dissolve cholesterol and fat and are good candidates to be used within the biocompatible solvent means.

### BRIEF DESCRIPTION OF DRAWINGS

Table 1: HSP parameters of select solvents and their ability to dissolve cholesterol and or fat.

FIG. 1: Illustration showing the mechanism by which the plaque is dissolved by the droplets of the biocompatible solvent means travelling through an artery

FIG. 2: Dispensing system for the biocompatible solvent means delivering solvent to an artery with the filtration and precipitation means providing blood filtration.

FIG. 3: Block diagram of the filtration and precipitation means.

FIG. 4: Second implementation of a filtration and precipitation means.

FIG. 5: Third implementation of a filtration and precipitation means with

FIG. 6: Simplified implementation of filtration and precipitation means.

### DETAILED DESCRIPTION OF PREFERRED EMBODIMENT

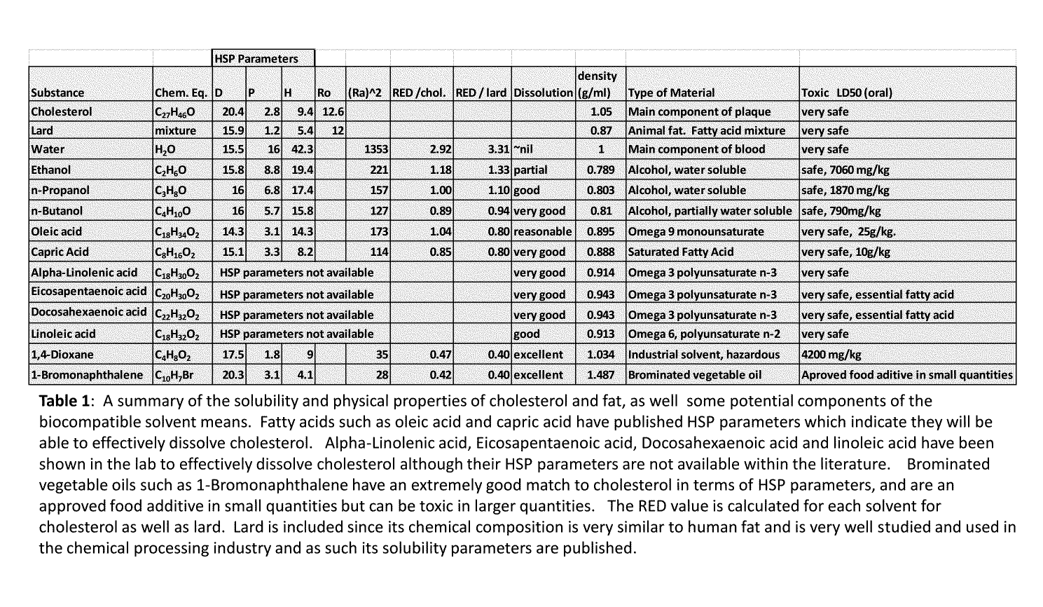

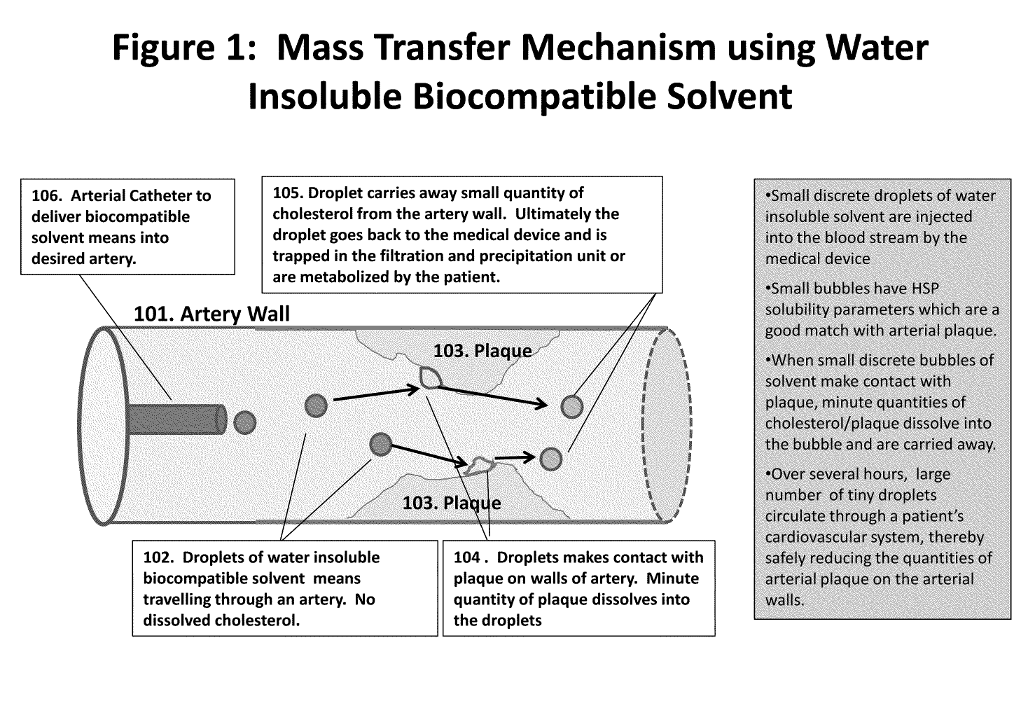

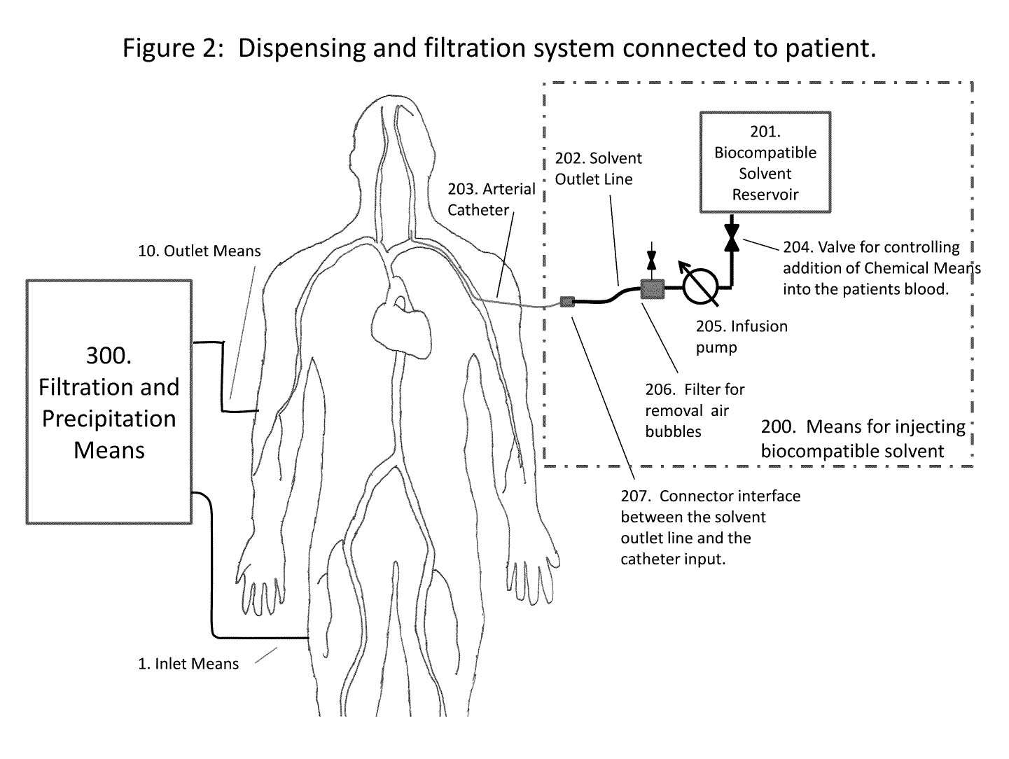

FIG. 1 is a pictorial highlighting the underlying principle of our patent. The small droplets of the water insoluble biocompatible solvent means 102 are injected into the blood stream by catheter 106 and travel through a patient's arteries 101. When they make contact with arterial plaque 103, given that they are an effective solvent for plaque and cholesterol, small amounts of the arterial plaque will dissolve into the droplet 104. The droplet which now has a small quantity of dissolved cholesterol 105 or other lipids from the plaque will then be washed downstream by the flowing blood thereby carrying the dissolved cholesterol or plaque through the blood stream until it is eventually drawn into the medical device where it will be trapped in the separation column 37, or combine with albumin or other lipoproteins and eventually be metabolized by the patient. Over several hours, thousands of these tiny droplets of the biocompatible solvent will travel through a patient's cardiovascular system and safely reduce the quantities of arterial plaque in a patient's cardiovascular system. This system can potentially offer a systemic method of treating the build up of plaque in all blood vessels, safely and with minimal side effects. Alternatively by the use of a catheter placed directly in an artery with a known blockage or buildup of plaque, it can provide a very focused treatment to the artery in question as well as all downstream branches and provide an alternative to angioplasty. One of the primary benefits of this method compared to angioplasty is that the plaque is actually removed and there is no damage to the blood vessel since it does not need to be stretched or kept open with a stent. Furthermore, in addition to treating one area, the droplets of the biocompatible solvent will treat all accumulations of plaque which are downstream from the catheter which is injecting the droplets of solvent. A third advantage is that this method could be used to treat arteries deep in the brain, or very small arteries which are currently not accessible using angioplasty. Finally, it is important to mention that the droplets of biocompatible solvent will continue to dissolve arterial plaque until they are metabolized, combined with blood proteins, or are drawn into the medical device (if a filtration and precipitation means is being used) and filtered.

The biocompatible solvent should preferably be insoluble in water and blood and have solubility parameters which are well matched to those of cholesterol, cholesterol esters or other fatty material we are trying to remove, be reasonably non-toxic in the quantities we need to administer and preferably have a density which is lower than blood to make it easy to remove from the patient's blood flow. Some of the most promising materials tested to date are fatty acids, or mixtures of fatty acids as discussed previously. The fatty acids which have been tested and show very promising results are a) Alpha-linolenic acid, b) Linoleic acid, c) Oleic acid, d) Elcosapentaenoic acid also known as EPA Omega3, e) Docosahexaenoic Acid also known as DHA Omega 3, f) Capric acid which is a medium chain saturated fatty acid. In principle any fatty acid or combination of fatty acids which have solubility parameters which are well matched to cholesterol and can be administered intravenously could be used. The fatty acids could be saturated, monounsaturated or polyunsaturated. Furthermore, they could be a short chain fatty acid, a medium chain fatty acid, a long chain fatty acid or even a very long chain fatty acid. Fatty acids are ideal for use or inclusion in the biocompatible solvent means since they are water insoluble, are safe for humans, can be completely metabolized by the patient and can have solubility parameters which are reasonably well matched to cholesterol. Furthermore, given that humans already eat large quantities of these fatty acids the potential long term health risks are very minimal. However, other organic molecules could be found to offer similar benefits and used for the same purpose without deviating from the spirit of the invention.

The ideal biocompatible solvent should have Hansen Solubility Parameters which are well matched to cholesterol or cholesterol esters to allow a sufficiently large quantity of plaque to be dissolve for a given quantity of biocompatible solvent, and have a calculated Relative Energy Difference (RED value) of less than 1.3, but preferably less than 1.0 and ideally less than 0.9 or even 0.8. Table 1 shows a few of the compounds which have been shown experimentally to be effective at dissolving cholesterol. A mixture of several compounds can be created where the Hansen Solubility Parameters of the mixture is a better match to those of cholesterol than any of the individual compounds. For example, at the bottom of Table 1, we show the Hansen solubility parameters for a mixture of 55% Capric acid, 15% Ethanol and 30% purified lard. Although the RED value for each of these compounds against cholesterol is 0.8, 1.18 and 0.84 respectively, the RED value of the mixture is 0.79. As such, mixtures of multiple compounds can be created to more closely match the solubility parameters of cholesterol to arrive at a very effective biocompatible solvent.

The other important figure of merit for the biocompatible solvent, which is related to the RED value, is the mass fraction of cholesterol or cholesterol esters, that can be dissolved into the solvent. In general, the lower the RED value the greater the mass fraction of cholesterol which will be dissolved into the biocompatible solvent. Based on lab results, a solution which has a RED value of about 1.2, will be able to dissolve a mass fraction of cholesterol of approximately 2%. A solution which has a RED value of 1 will be able to dissolve a mass fraction of cholesterol of approximately 6%. Finally, a solution which has a RED value of 0.9 was shown to be able to dissolve a mass fraction of approximately 7%. Conversely, a solvent with a RED>>1 will dissolve very little solvent, for example an RED value of 2.9 such as water, will only dissolve 95 micrograms per litre, which corresponds to a mass fraction of 9.5×10−8 or 0.0000095%. The ideal biocompatible solvent will be able to dissolve a relatively large mass fraction of cholesterol. A solution of the biocompatible solvent at 37° C. will ideally be able to dissolve a mass fraction of at least 1% cholesterol, but preferable greater than 5% and ideally greater than 10% to ensure we are able to dissolve the greatest quantity of plaque for the smallest amount of biocompatible solvent. Of course the toxicity of the biocompatible solvent vs. the solubility is also important and it may be better to chose a biocompatible solvent with a lower solubility but very few if any side effects.

The list of saturated fatty acids is as follows Propionic acid (Propanoic acid), Butyric acid (Butanoic acid), Valerie acid (Pentanoic acid), Caproic acid (Hexanoic acid), Enanthic acid (Heptanoic acid), Caprylic acid (Octanoioc acid), Pelargonic acid (Nonanoic acid), Capric acid (Decanoic acid), Undecylic acid (Undecanoic acid), Lauric acid (Dodecanoic acid), Tridecylic acid (Tridecanoic acid), Myristic acid (Tetradecanoic acid). For saturated fatty acid molecules larger than Lauric acid the melting temperature increases above the normal core body temperature of a patient. For example, Lauric acid with 12 carbon atoms has a melting temperature of 43° C. Myristic acid with 14 carbon atoms has a melting temperature of 54° C. Since the melting temperature of these larger saturated fatty acid molecules gets too high and it would be in solid form at 37° C. they would offer limited usefulness as the main component of the biocompatible solvent. They could nevertheless be used in small quantities if dissolved in another type of saturated fatty acid which is in liquid form at 37° C.

Unsaturated fatty acids have a lower melting temperature for a comparable molecular size and as such larger molecules could be used while remaining liquid at a normal core body temperatures of 37° C. For example Docosahexaenoic acid (DHA Omega 3) is a large molecule with 22 carbon atoms while the melting temperature is −44° C. The list of unsaturated fatty acids includes: α-Linolenic acid (ω-3), Stearidonic acid (ω-3), Eicosapentaenoic acid (ω-3), Docosahexaenoic acid (ω-3), Linoleic acid (ω-6), γ-Linolenic acid (ω-6), Dihomo-γ-linolenic acid (ω-6), Arachidonic acid (ω-6), Docosatetraenoic acid (ω-6), Palmitoleic acid (ω-7), Vaccenic acid (ω-7), Paullinic acid (ω-7), Oleic acid (ω-9), Elaidic acid (ω-9), Gondoic acid (ω-9), Errucic acid (ω-9), Nervonic acid (ω-9) and Mead acid (ω-9).

The biocompatible solvent means can be administered stand alone as a regular injection to perform a daily or weekly treatment. In this scenario, the injection could be directly in a vein. Once in the vein, the droplets of biocompatible solvent would be drawn back towards the heart and be broken up into much smaller droplets by the pumping action of the heart. The small droplets would then be pushed into the arterial system where they would collide and come into contact with plaque in the various arteries, thereby dissolving small quantities of plaque and helping to reduce or reverse atherosclerosis in the patient. This type of treatment would be administered over an extended period lasting many weeks or even years to provide a systemic treatment and a gradual reduction in arterial plaque in the entire cardiovascular system. If excessive amounts of the fatty acid are injected into the patient and the individual is feeling unwell, an injection of human albumin could be administered to bind with the fatty acid and allow it to be rapidly metabolized.

The quantity of biocompatible solvent which is injected and the rate at which it is injected are also important. On average, a human has about 5 litres of blood, of which 55% is blood plasma, and of this approximately 6-8% is blood proteins. As such, a typical human will have approximately 165 to 220 ml of blood protein within their cardiovascular (one word) system. The quantity of blood protein per 1 ml of blood is approximately 42 mg⁷. The albumin molecule is relatively large and has an approximate weight of 66437 atomic mass units (AMU). As such, in 1 ml of blood there would be approximately 6.3×10⁻⁷ moles of albumin protein. For comparison, a small droplet of DHA Omega3 fatty acid, with a molecular weight of 328 AMU would contain approximately 2.9×10⁻³ moles per ml. As such, given the finite rate at which albumin diffuses through the blood, and the fact that the droplet contains ˜4500 times as many fatty acid molecules as there are albumin molecules in the surrounding 1 ml of blood, a very small percentage of the droplet would be solubilized (checked) by the blood proteins within the short time it would take the droplet to travel from the output of the needle or catheter to an artery which contains arterial plaque. A smaller droplet measuring 1 mm per side, would only contain approximately 2.86×10−6 moles of fatty acid molecules, which is only 4.5× as many molecules as there are albumin molecules in the surrounding 1 ml of blood and such a small droplet would be solubilized more quickly. Finally, a very small droplet of fatty acid measuring 100 microns per side would contain approximately 4.5 nanomoles of fatty acid molecules. In this scenario the surrounding 1 ml of blood contains 220 times more albumin molecules as there are fatty acid molecules within this tiny droplet, and we would expect the droplet to be absorbed by the albumin in relatively short order. Depending on the number of binding sites of albumin, as well as the rate at which the albumin particles diffuse through the blood and come into contact with the droplet of biocompatible solvent, a larger droplet may be beneficial to ensure it reaches the targeted arterial plaque prior to being solubilized. The size of the droplets needs to be sufficient to ensure that a sufficiently small fraction of the droplet volume dissolves into the blood prior to making contact with arterial plaque and having an opportunity to dissolve the plaque. Ideally less than 1% of the droplet should be solubilized prior to making contact with the arterial plaque but in practise if the droplets are small, or we are injecting the biocompatible solvent into a vein as opposed to directly into an artery, a larger proportion of the biocompatible solvent may be dissolved by blood proteins. Nevertheless, even if a very small fraction of the droplet makes contact with plaque, >1% (99% of the droplet is solubilized), the treatment will still be effective but may require multiple sessions. Ideally, we would like to see less than 10% of the biocompatible solvent be dissolve into the blood, or solubilized by blood proteins as it travels from the entry point to the arterial plaque, but the system would nevertheless give reasonable results if as much as 90% of the biocompatible solvent is dissolved or solubilized.

It is important to reiterate that under normal circumstances the concentration of unbound free fatty acids which are circulating in human blood is very slow, especially the long and very long chain fatty acids which have solubility parameters which are well matched to those of cholesterol. A study conducted by Goodman⁸, concluded that the concentration of unbound fatty acids in bodily fluids is on the order of 10 nanomolar. As such, in 1 litre of blood we would expect approximately 10⁻⁸ moles of fatty acid molecules, and assuming an average molecular weight of 328 AMU this would correspond to about 3.28 micrograms per litre, or 3.28 nanograms per ml. It is interesting to note that this number is actually smaller than the solubility of cholesterol in pure water, which is approximately 95 micrograms per litre at 30° C. The main reason for the difference is that in the case of blood, the fatty acids are not allowed to dissolve until the saturation concentration is achieved, but rather bind with blood proteins and are removed from circulation as they become available. As such, the concentration of free fatty acids in human blood is actually much lower than in pure water, due to the effectiveness with which albumin blood protein binds with the free fatty acid molecules.

Ger J. van der Vusse provides an estimate for the speed at which albumin can transport fatty acids through the blood stream to adipose tissue⁶. The average human contains approximately 210 g of albumin. This corresponds to approximately 0.003 moles. The transport efficiency is approximately 0.25 per capillary pass, which is to say that an albumin molecule has approximately a 25% probability of transferring a fatty acid molecule to an adipose tissue cell (or other cell) per capillary pass. On average, it takes blood approximately 1 minute to make a complete loop through the circulatory system. As such, assuming there are 1-2 free fatty acid molecules bound to each albumin molecule, fatty acids can be solubilized at a rate of about 0.0008 moles per minute, or approximately 0.2 to 0.4 g/minute. This provides an estimate order of magnitude of the speed with which the blood proteins can bind with the biocompatible solvent and solubilize the droplets. Based on this approximation, if one were to provide a small injection of 10 ml of a biocompatible solvent containing fatty acids, it would take anywhere from 25 to 50 minutes for the droplets to be fully solubilized by the blood proteins and during this time they would be travelling through the cardiovascular system and dissolving small quantities of arterial plaque. A second parameter which needs to be considered is to understand the total quantity of fatty acids which can be bound by albumin in a typical human. As mentioned previously, the average human has about 210 g of albumin which corresponds to 0.003 moles. Assuming each albumin molecule binds 2 fatty acid molecules, we have the potential to bind and solubilize 0.006 moles of fatty acid at any given moment. For Oleic acid with a molecular mass of 282 AMU this corresponds to approximately 1.69 grams of Oleic acid, or 1.89 ml, based on a density of 0.895 g/ml. This is an important consideration since it if we inject a quantity of fatty acid in excess of this amount, even if every albumin molecule in a patients circulation were to come into contact with the biocompatible solvent we could at most solubilize approximately 1.89 ml of the Oleic acid. Any excess amount would need to be gradually solubilized as the albumin transfers its bound fatty acid payload to the various cells of the body during the capillary pass, and this would occur at a rate of approximately 0.2 to 0.4 grams/min.

If a very small quantity of biocompatible solvent is injected at a very slow rate, the biocompatible solvent may combine with albumin or other blood proteins before it has the opportunity to come into contact with arterial plaque and the effectiveness of the treatment will be reduced. For example, if we administer very small droplets at a rate which is inferior to 0.2 grams/min it is conceivable that all the biocompatible solvent will be solubilized prior to having an opportunity to dissolve arterial plaque. Conversely, if a small quantity of biocompatible solvent is injected very rapidly into a vein or artery, for example 5 ml within a few seconds, a few large droplets of the solvent will travel through the cardiovascular system and make contact with arterial plaque and dissolve a small quantity of the plaque, before the albumin has the opportunity to bind with the solvent. Based on the estimate provided by Vusse it could take nearly 12-25 minutes for a 5 ml injection to be solubilized. To ensure the treatment is effective, the size of the droplets needs to be sufficient to ensure that a fairly small amount of the droplet dissolves into the blood prior to making contact with arterial plaque and have opportunity to dissolve the plaque.

A third approach can be to administer a sufficient quantity of biocompatible solvent such that a majority of the binding sites of the albumin and other lipoproteins have been used up, and then any excess amounts of biocompatible solvent will continue to travel through the circulatory system for an extended time period thereby effectively dissolving arterial plaque. As mentioned previously, a rough order of magnitude is that the albumin can bind with approximately 0.006 moles of fatty acid molecules at a given instant, which corresponds to approximately 1.7 grams of Oleic acid. As such, injecting 2 ml, or 5 ml or 10 ml or 20 ml of fatty acid in a short period of time would result in droplets of the fatty acids circulating freely within the cardiovascular system and making contact with arterial plaque. After a prescribed time period, if necessary, the patient can be given an injection containing blood proteins such as albumin to allow the excess amounts of the biocompatible solvent to bind with the blood proteins and be solubilized within the blood. Alternatively, the patient's own albumin can be allowed to bind with the fatty acids in the biocompatible solvent and transferred to adipose tissue. A third approach can be to use the filtration and precipitation means which will be described shortly to systematically remove the excessive amounts of biocompatible solvent. For a more focused and rapid treatment when a patient is known to have a large accumulation of plaque in a specific artery, the biocompatible solvent means can be injected directly into a target artery using Means for Injecting biocompatible solvent 200. The means for injecting the biocompatible solvent 200 could be connected to an arterial catheter 203 to deliver the biocompatible solvent directly upstream of the arterial plaque or arterial system needing treatment. The surgeon can place the tip of the catheter in the target artery, just slightly upstream of the plaque we desire to dissolve using a fluoroscope and the standard methods currently used by cardiologists and other medical professions. The infusion pump 205, or similar device, can then be used to send a flow of solvent into the artery. The infusion pump 205 would draw biocompatible solvent form the biocompatible solvent reservoir 201. The Means for injecting the biocompatible solvent 200 would also include valves 204 and filter to remove air bubbles 206 as is generally known in the art for systems used to inject drugs or saline intravenously into patients. Not shown but also potentially required would be a bleed valve to remove air bubbles and bleed the line after the arterial catheter 202 and solvent outlet line 203 are joined by connector 207. The flow rate of the solvent should be lower than the flow rate of blood through this same artery to ensure the primary function of the artery, which is to deliver oxygen rich blood to the downstream tissue, is not impaired. For example, if the artery we are treating has a typical flow rate of 50 ml/min, we would set the infusion pump to a flow rate which is considerably less than this, say approximately 5 ml/min. Over the course of 1 hour, we would have injected 300 ml of solvent into the patient, and through this artery, which is sufficient to dissolve several grams of plaque. This is very high flow rate and would only be feasible if we are also using a Filtration and Precipitation Unit 300 to systematically remove droplets of the biocompatible solvent from circulation to allow new solvent to be injected. If the Filtration and Precipitation Unit 300 is not being used, we should avoid injecting the patient with excessive amounts of biocompatible solvent, significantly in excess of what the patient's blood proteins can solubilize and his/her metabolism can absorb.

If small flow rates are being used to minimize the amount of biocompatible solvent which is injected into a patient, to prevent the droplets of solvent from binding with the albumin before they have an opportunity to come into contact with the plaque, it may be beneficial to modulate the flow rate of the pump in an on/off manner. For example, if a small artery is being treated which would normally receive 50 ml/min of blood flow, and we wish to administer 1 ml/min of solvent, it may be better to inject a small burst of 0.25 ml several times per minute, rather than have a continuous flow of 1 ml/min or 0.017 ml/sec. If a small droplet measuring 0.008 ml exits the tip of the catheter twice per second, this very small droplet will have a relatively large surface area to volume ratio, and could be rapidly solubilized by the blood proteins. Conversely, if the pump is modulated and a large droplet of 0.25 ml every 15 seconds, this droplet will have a much smaller surface area to volume ratio and the blood proteins will not have an opportunity to solubilize the biocompatible solvent prior to reaching the arterial plaque being treated.

In this scenario, if we are not using a filtration and precipitation unit, we must rely on the patient to metabolize the biocompatible solvent. If we are using a fatty acid, after some time the solvent will bind with albumin or other lipoproteins and be metabolized. If additional amounts of biocompatible solvent are needed and administered via the catheter, the patient can be given an injection of human albumin to allow additional these amounts of the fatty acid to be metabolized. It is important to mention that the albumin would need to be injected by another access point, and not through the catheter which is being used to deliver the biocompatible solvent means into the artery. The biocompatible solvent must be allowed to circulate as discrete droplets for a short time, to ensure they can come into contact with the arterial plaque and dissolve the cholesterol or other components of the plaque, before they are gradually dissolved and broken down by the albumin and other blood proteins. By injecting albumin, additional amounts of the biocompatible solvent can be administered and metabolized. Injecting albumin is not strictly necessary and the patient's own blood proteins will gradually solubilize the biocompatible solvent.

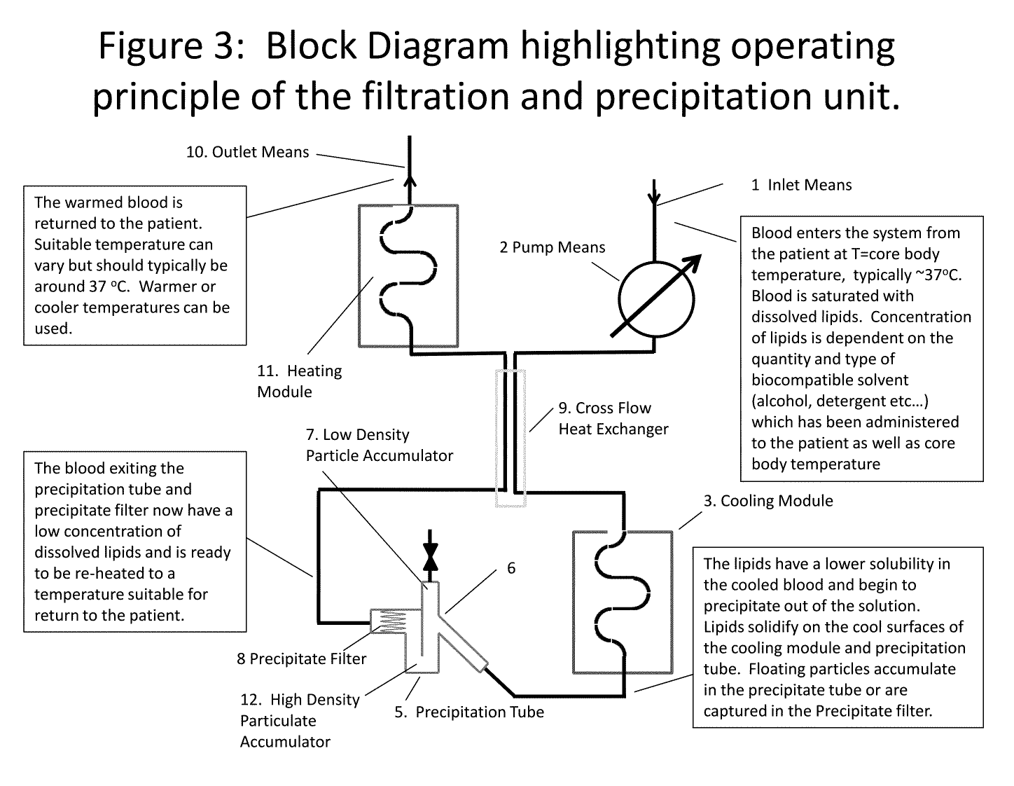

Alternatively, if we use a filtration and precipitation means 300 we can systematically remove the droplets of biocompatible solvent form the patients circulatory system and they need not all be metabolized. By using the precipitation and filtration means, additional amounts of the biocompatible solvent means can be injected into the artery to dissolve the arterial plaque being targeted, and then systematically remove it from the circulatory system using the filtration and precipitation means 300. Preferred embodiments of the filtration and precipitation means 300 are described below in detail and shown in FIGS. 3, 4, 5 and 6.

A block diagram explaining the operating principle of the precipitation and filtration means 300 can be found in FIG. 3. The precipitation and filtration means can be any device, system or method which is known in the art to be effective in removing low density droplets of a fatty acid or other water insoluble compounds from human blood. The precipitation and filtration means can be based on density, solubility, physical or chemical characteristics of the biocompatible solvent means and should safely remove the solvent without contaminating the blood or damaging the blood cells in a way that would have adverse effects for the patient. In this implementation the system cools the blood to help achieve a separation of the biocompatible solvent means from the blood stream. The system is connected to a patient through an input means which can comprise of an input catheter or needle and or an intravenous line which is connected to the input line 1. The input means can be connected to any suitably sized vein or artery capable of supporting the desired flow rates. A pump means 2 is used to draw blood through the input catheter into the input line 1. The pump means 2 can be any type of pump (check font size??? and remember to change to one font) which is suitable for processing blood, such as a peristaltic pump, centrifugal pump, positive displacement pump or continuous flow pump. The pump then pushes the blood into an optional cross flow heat exchanger 9, which is used to transfer heat between the incoming blood flow, and the exiting blood flow to reduce the power and size requirements of the cooling and heating systems. The cross flow heat exchanger is designed by thermally coupling the inflow tube with the outflow tube and having the two flows move in opposite directions through the heat exchanger as is known in the art. If properly designed, a majority of the heat from the incoming blood flow can be transferred to the outgoing blood flow. The cross flow heat exchanger serves to reduce the power requirement of the cooling and heating system but the system could be made to work without the cross flow heat exchanger. The blood then flows into the cooling and precipitation unit 3. The cooling and precipitation unit 3 consists of a heat exchanger 4, which can be used to further cool the blood or to maintain the blood at a cooled temperature and cause the biocompatible solvent mean droplets to solidify and be trapped in the filtration unit, or alternatively float to the top of the low density particle accumulator 7. The droplets of the non water soluble biocompatible solvent contain dissolved arterial plaque, cholesterol, cholesterol esters and other fatty compounds. Some material may precipitate and form liquid or solid particulates in the blood whereas other material may solidify onto the walls of the heat exchanger or filter unit. If using a water insoluble solvent with HSP parameters which are well matched to the cholesterol or lipid which is being transported, a majority of the material will remain dissolved in the solvent droplets and be removed from the patient's circulatory systems as the solvent is separated from the blood flow. The blood then passes through a precipitation and filtration unit 5, which removes liquid and solid particulates of the non water soluble solvent from the blood stream. The walls of the precipitation and filtration unit 5 are kept at a lower temperature to favour the solidification of the droplets or other lipids onto the walls of the unit. Given that the solvent droplets which are carrying the dissolved lipids preferably have a lower density than blood, they can be removed by allowing them to float to the top in a long slanted column 6 or be separated using a centrifugal system and are trapped and accumulated in the Low Density accumulator 7. This trap collects both liquid and solid particulates which have a lower density than blood. Cholesterol particulates have a density of 1.05 g/ml which is very similar to that of whole blood and will not float to the top of the column. However, the cholesterol which is dissolved in the droplets of the biocompatible solvent are trapped in the droplet and will be accumulated in the Low Density accumulate 7. Solid particles which fail to float upwards, can be removed using a filter means 8, to impede their progression. The filtration means is designed to allow blood plasma and blood cells to pass while trapping solid particulates of the water insoluble solvent, cholesterol or lipids which are floating in the blood or large droplets of the water insoluble solvent. The filtration means can be based on the physical size of the particles or leverage the hydrophobic characteristics of the biocompatible solvent. The blood is then re-heated to a temperature near 37° C. in the cross flow heat exchanger 9. Optionally, a secondary heater can be used to further increase the temperature of the returning blood flow to a desired temperature which can be greater or less than 37° C. The blood with a lowered level of dissolved cholesterol, cholesterol esters or fatty material is sent to Outlet Means 10 such as a catheter, needle or intravenous line installed on any suitably sized vein or artery and returned to the patient. The device can be designed to be a continuous flow apparatus where blood continually flows in and out of the system, or a controlled volume type design where blood is repeatedly taken and processed in batches and then re-inserted into the patient, potentially from the same access point. The preferred embodiment shown here is a continuous flow type device.

Not shown in the diagram are the cooling capability and a heating capability, as well as a control module which regulates the temperature and blood flows and monitors the temperature. The cooling can be provided by several methods such as by passing chilled water through a heat exchanger, using a refrigeration unit, thermo-electric heaters/coolers or a heat pump. Similarly, the heating can be provided using an electric heater, a heat pump, passing warmed water through a heat exchanger in contact with the unit or blood or thermo-electric heaters.

The cross flow heat exchanger 9 is very effective in reducing the power requirements. Given that we are taking warm blood at about 37° C., cooling it down to a desired temperature to cause precipitation, and then re-heating it back to approximately 37° C., the device can be designed to form a cross flow heat exchanger to reduce power requirements. In this type of implementation, the cool blood which is exiting the apparatus is caused to flow in a tube, which is in good thermal contact to a second tube which is carrying warm blood into the apparatus. The cool blood which is exiting the apparatus, is used to cool the warm blood which is entering the apparatus. A cross flow design is preferred for this implementation but not necessary. Although more expensive and complicate, more effective filtration could be implemented by using a centrifuge to accelerate the separation of liquid and/or solid particulates of the biocompatible solvent or fats from the blood stream.

The required flow rates are fairly modest, and can be in the range from as low as 10 ml/min to as high as 500 ml/min or even higher. There is no strict minimum or maximum range. If the range is too low, relatively low transport rates will be achieved and greater quantities of the biocompatible solvent will need to be metabolized by the patient. Conversely, if the blood flow is too high, the cost and size of the instrument will increase with little benefit to the patient other than that the treatment time will be reduced. A secondary complication of using excessively high flow rates is that the catheters would need to be inserted into large blood vessels which are capable of supporting such large flow rates. Also, the flow rates should be kept low enough to allow the biocompatible solvent drops which carry the cholesterol to be separated from the blood flow in the Precipitation and Filtration Unit 5. Furthermore, to keep the procedure simple, it is desirable to insert the catheters into veins. However, if excessively large quantities of blood are drawn from a vein, the vein can collapse and restrict further blood flow. Arteries have rigid walls and as such can support larger flow rates, but inserting a catheter into an artery is a more complicated and risky procedure. For these reasons, using lower flow rates with a longer treatment duration may be the preferred and simpler approach.

This type of procedure or device would find many applications. It could be used as a periodic treatment to prevent or reverse atherosclerosis or to treat obesity or other disorders which are caused by elevated lipid levels in a person's metabolism. For patient's which have had a heart attack or stroke, or are considered to be in imminent risk of having a heart attack or stroke, the patient could be admitted to the hospital, and a prolonged treatment lasting several days could be initiated to rapidly reduce the accumulated plaque from a patient's arteries. The treatment is very minimally invasive and requires only two catheters to be inserted to allow a blood inlet and outlet into the apparatus, if using a continuous flow system. If using a control volume system which processes blood in batches, then a single catheter could be used since blood can be drawn from the catheter, processed, and then reinserted through the same catheter. The blood vessels can be any vein or artery which is suitably sized to support the desired flow rates through the apparatus. Preferably, the inlet and outlet should not be inserted into the same blood vessel to prevent the fluid from circulating directly from the outlet to the inlet since this would significantly reduce the effectiveness of the filtration and precipitation unit.

For a patient which is known to have an artery with restricted blood flow due to arterial plaque, the device could be used in conjunction with an arterial catheter to provide a more focused treatment to a specific artery. The output line 10 would be connected to an arterial catheter. The output of the arterial catheter could be placed in the artery and deliver warmed blood with a low concentration of cholesterol and saturated fats in addition to the biocompatible water insoluble solvent, directly to the blood vessel in question, slightly upstream of the plaque which needs to be dissolved. For this type of procedure, having blood exiting the catheter at a slightly higher temperature than 37° C., is beneficial since it will help dissolve the plaque which is occluding the artery in question. The secondary heater 11 could be set to heat the blood to a temperature above 37° C. C and potentially as high as 49° C. with little to no damage to the blood cells to further accelerate the dissolution of the arterial plaque in the target artery. Furthermore, the water insoluble biocompatible solvent means would be injected into the warm blood stream which is being directed to the target artery to accelerate the dissolution of the plaque. The catheter can enter the body via the femoral artery (in the leg) or another suitable artery, and guided to the desired treatment location using a fluoroscope in a similar manner to that used to administer angioplasty. For this type of a procedure, the input line and input catheter must also be from an artery to ensure oxygenated blood is delivered to the artery which is being treated to ensure the downstream tissue is not deprived of oxygen. The medical device used in this way, could be a substitute for angioplasty by allowing the targeted treatment of plaque build up in certain arteries.

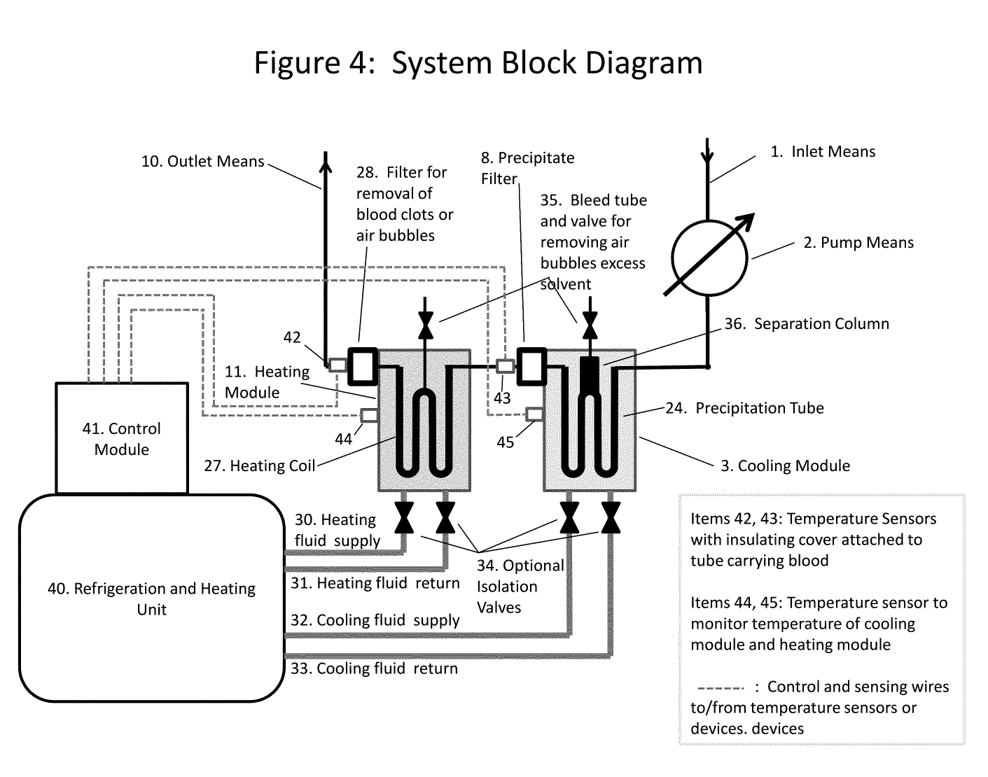

A more detailed system block diagram of a preferred implementation of the filtration and precipitation unit which includes a control system can be seen in FIG. 4. This version of the preferred implementation does not include a cross flow heat exchanger. Blood can enter from the patient via Intel Means 1. The inlet means can include a catheter, needle or IV line which draws blood from any reasonably sized artery or vein from a patient. The blood then enters pumping means 2. Pumping means 2 can be any type of pump which is known in the art to be suitable for use in an extra-corporeal blood circuit such as a peristaltic pump, centrifugal pump, positive displacement pump or continuous flow pump. The blood rich is saturated lipids and with droplets of the biocompatible solvent means then enters Cooling Module 23, where it undergoes a temperature reduction in Precipitation Tube 24. The tube preferably flows in an up/down pattern to allow for trapping of low density and high density lipid particles which precipitate out of the blood as it is caused to cool. The precipitation tube can incorporate any precipitation means which is known in the art to be an effective method of separating low density particles from a fluid flow of a higher density, and which is suitable for processing blood. The blood can be cooled to a temperature approaching 0° C. Furthermore, droplets of the biocompatible solvent means can be trapped in the separation column 36. As the blood cools, the solubility of dissolved lipids decreases and the lipids precipitate. Some droplets of the water insoluble biocompatible solvent and lipids may solidify on the walls of the Precipitation Tube 24 while others form solid particles which float in the solution and are eventually trapped by Precipitation Filter 8 or are trapped in the separation column 36. A temperature sensor 43 which can be wrapped around the tube at the exit of the Cooling Module 23, is used to monitor the temperature of blood exiting the cooling module. The sensor does not come into contact with the patients blood and is therefore reusable. To ensure an accurate reading, the sensor is a thin film device which can be wrapped onto the outside surface of the tube and is then wrapped with an insulating cover to ensure the reading is not altered by the ambient air temperature. Similarly, temperature sensor 42 allows the control module to monitor the temperature of blood exciting the Heating Module 11. The blood exiting the cooling module 3 should now have a relatively low concentration of dissolved lipids and a majority of the biocompatible solvent removed

The blood then enters the Heating Module 11 and flows through the Heating Coil 27 where it is brought back up to a relatively warm temperature suitable to be returned to the patient. The temperature at the exit of the heating module should be between 30° C. and 49° C., more typically 37° C. so that it neither cools or heats the patient. 49° C. would be the upper limit since blood cells are known to be damaged beyond this temperature. It is not absolutely necessary to reheat the blood before returning it to the patient if small flow rates are used and if the blood is not being directed directly to an artery being treated. If the Outlet Means 29 is connected to a catheter which has been placed in an artery where a surgeon is attempting to dissolve plaque, the exit temperature can be raised to a temperature slightly above core body temperature, but not so high that the blood cells are damaged, somewhere between 37° C. and 49° C., to favour the dissolution of the plaque being treated.

A programmable Refrigeration and Heating Unit 40 is used to control the temperatures of the heating module 11 and cooling module 3. The Refrigeration Unit in 40 maintains a reservoir or cooling fluid at a desired temperature and this cooled fluid is pumped to the cooling module 3, via tubes 32 and 33. Tube 32 carries cooling fluid at the desired temperature to cooling module 3 while tube 33 returns the cooling fluid back to the Refrigeration and Heating Module 40. Similarly the heating module in 40 maintained a reservoir of heating fluid at a desired temperature and this heating fluid is pumped to Heating Module 11 to heat the blood to the desired temperature. The programmable heating and cooling module 40 includes two circulation pumps to cause the cooling and heating fluids to flow to cooling module 3 and heating module 11 respectively. The temperature of the cooling fluid and heating fluid in 40 are controlled by Control Module 41. Control module 41 monitors the temperature of the blood leaving cooling module 3 and heating module 11 using temperature sensors 43 and 42 respectively. Furthermore, the temperature of the cooling fluid in cooling module 3 is monitored using temperature sensor 45, and likewise the temperature of the heating fluid in heating module 11 is monitored using temperature sensor 44.

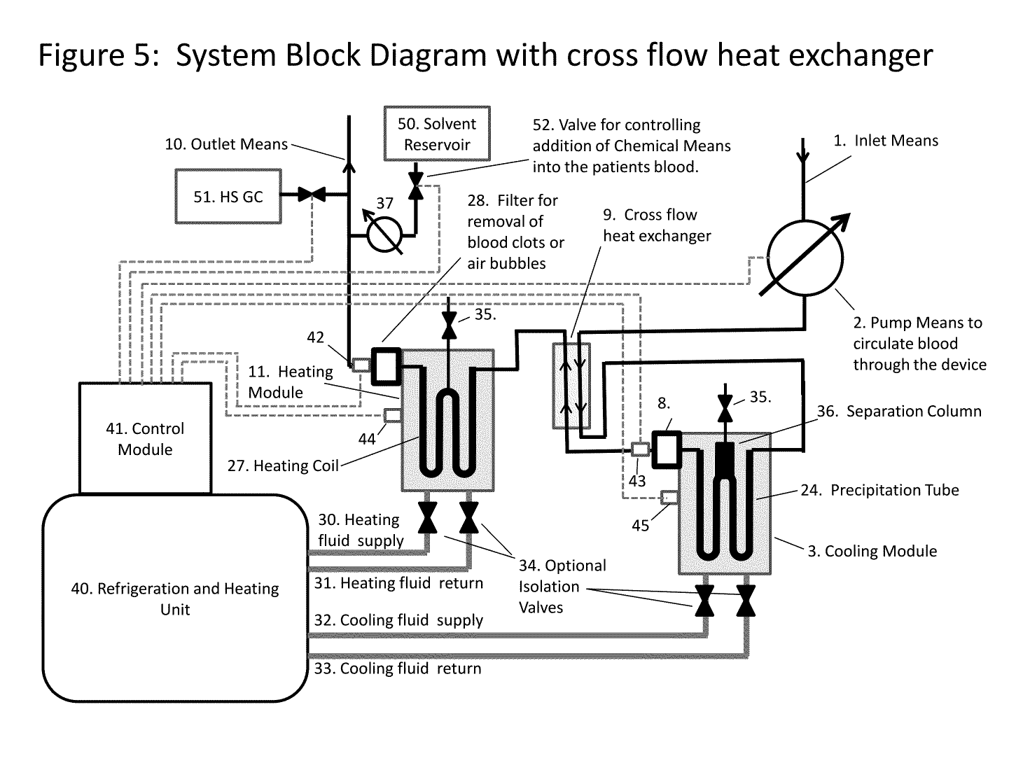

FIG. 5 shows a slight variation of the System block diagram. In this implementation we have included the optional Cross Flow Heat Exchanger 9 which reduces the power requirements of the heating and cooling system in Module 40. Furthermore, we have included a Means for Analyzing the concentration of solvent in the patient's blood stream such as a Head Space Gas Chromatograph. The Head Space Gas Chromatograph 51 can analyze the concentration of alcohols or other liquids in the patient's blood stream by measuring the partial pressure of the gases in the head space above the blood sample. For prolonged treatments this device could periodically take a small blood sample and feed the results back to the Control Module 41 to allow more or less of the biocompatible solvent means to be released into the patient's blood stream. Alternatively, the blood sample could be taken manually by a medical professional overseeing the treatment and inserted into the HS-GC, or some other means of analysis. A Head Space Gas Chromatograph is but an example of one of the means which could be used to analyze the quantity of chemicals in the patients blood stream.

Also included in this diagram is a Means of Adding the biocompatible solvent means to increase the solubility of lipids in the blood stream such as Alpha-linolenic acid also known as Omega 3, Linoleic acid also known as Omega 6, oleic acid also known as Omega 9, Elcosapentaenoic acid also known as EPA Omega3, Docosahexaenoic Acid also known as DHA Omega 3, 1,4 dioxane., Capric acid, 1-bromonaphthaline or any other biocompatible solvent which may be identified. The biocompatible solvent means could be added via an IV drip, where the valve is either controlled automatically by the control module, or manually by a medical professional overseeing the treatment. Furthermore, a pump means 37 can be used to pump the water insoluble solvent means into the blood stream to overcome the positive pressure.

The elements which come into contact with the patient's blood form part of a disposable set which is replaced with every treatment. In this system, the disposable set comprises of Cooling Module 3, Heating Module 11, Precipitation Tube 24 and Heating Coil 27. The tubing which carries blood is also part of the disposable set.

Elements which are part of the medical device and can be reused with each treatment are the Control Module 41, Refrigeration and Heating Module 40, temperature sensors 42, 43, 44 and 45, as well as the pump 2. If a peristaltic pump is used, the blood does not come into contact with the actual pump and the pump can be reused, only the tube needs to be changed.

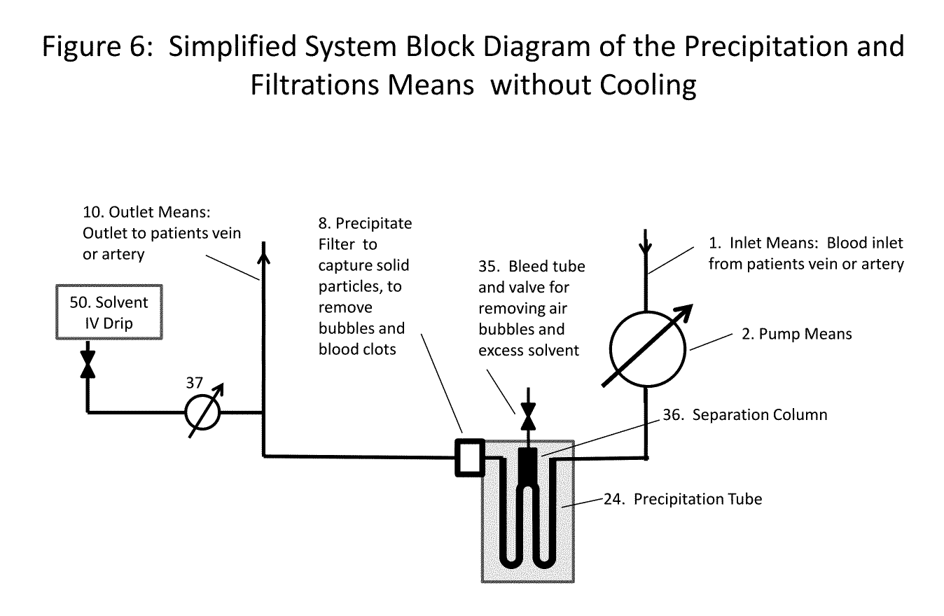

The use of a cooling system is desirable but not necessary. The biocompatible solvent means carrying the dissolved cholesterol or other undesirable lipids can be trapped in the filtration unit based purely on the lower density of the droplets and or their hydrophobic characteristics without the need for cooling. FIG. 6 shows such a system. The blood enters the precipitation tube 24 and the droplets of the biocompatible solvent means are trapped in the separation column 36 based purely on the fact that they have a lower density than blood. A solvent IV drip 50 and pump means 37 can be used to inject a controlled amount of the water insoluble solvent means into the blood stream just prior to being re-injected into the patient. A bleed valve 35 can be used to remove the accumulated water insoluble solvent from the separation column 36 when sufficiently large quantities have been trapped and the column is nearly full.

### Alternative Embodiments

The preferred embodiment of the invention has identified biocompatible solvents which are safe for use in humans and also able to dissolve cholesterol and fatty compounds of which arterial plaque is composed. We have also shown that water insoluble biocompatible solvents such as fatty acids offer great promise compared to water soluble solvents since they do not mix with the blood and little droplets of the biocompatible solvent maintain their solubility parameters as they travel through the blood and effectively dissolve arterial plaque upon making contact. Although a majority of the biocompatible solvents we have proposed are based on fatty acids, it should be understood that other chemicals could be used and developed to achieve a similar purpose. For example, any organic molecule which is reasonably insoluble in blood such that it can travel from the catheter or entry point to the arterial plaque we wish to dissolve, as a small droplet without dissolving into the blood, and has solubility parameters which are sufficiently well matched to cholesterol, could potentially be used. Most of our focus has been on fatty acids which are a subset of the Carboxylic acid family of organic molecules, primarily because they are safe and have been shown to be effective during our tests and based on our research of their solubility parameters. However, other types of organic molecules could potentially be used. It is conceivable that we could identify molecules from the Organometallic family, Nitro family, Nitrile family, Aminine family, Amide family, Carboxylic acid chloride family, Ester family, Ketone family, Aldehyde family, Carbonyl family, Ether family, Halide family, Arene family, Alkyne family or Alkene family. Alcohols could also be used. Given that Ethanol, Propanol and Butanol are water soluble they could not be used as the main component of the biocompatible solvent but could be used in small quantities to adjust the solubility parameters of the mixture. Also, longer chain alcohols which are not water soluble and found to have acceptable levels of toxicity could potentially be considered as one of the components of the biocompatible solvent.

### INDUSTRIAL APPLICABILITY

Apparatus, systems and methods are provided which allow cholesterol, cholesterol esters, and other fatty materials to be removed from the cardiovascular system of a patient to treat atherosclerosis, which is the main cause of heart disease and stroke. Biocompatible solvents have been identified which can effectively dissolve cholesterol and other fatty compounds of systematically reverse the accumulation of arterial plaque within a patient In addition, the apparatus and systems which allow the biocompatible solvents to be used have been described in detail. A means for administering the biocompatible solvent and a means for filtering the solvent form a patient's blood once administered have been described in detail. Given that heart disease and stroke remain the leading cause of death in the developed world, there is a great need for new technologies and ideas within the medical community and the inventions described herein should find widespread acceptance and use.

### REFERENCES

1. Wikipedia reference on Atheroma, http://en.wikipedia.org/Wiki/Arterial_plaque
2. Shipra Baluja et al., “Solubility of Cholesterol in some alcohols from 293.15 to 318.15K”, Archives of Applied Science Research, 2009, 1 (2) pp. 263-270.
3. “Hansen's Solubility Parameters a Users Handbook” Second Edition, by Charles M. Hansen, CRC Press
4. Cutnell, John & Johnson, Kenneth. Physics, Fourth Edition. Wiley, 1998: 308.
5. John McMurry, “Fundamentals of Organic Chemistry”, Brooks/Cole Publishing Company, 1986.
6. Ger J. van deer Vusse, “Albumin as Fatty Acid Transporter”, Drug Metab. Pharmacokinet. 24 (4):300-307, 2009.
7. Goodman, D. S.: “The Interaction of Human Serum Albumin with Long-chain Fatty Acid Anions”, Journal of American Chemical Society, 80: 3892-3898, 1958.

1. Use of a biocompatible solvent for treating or preventing heart disease, stroke or atherosclerosis characterized by:
a) Administering a biocompatible solvent means into a patient's blood stream such that small droplets of the said biocompatible solvent means travels through the cardiovascular system and dissolve small quantities of arterial plaque upon making contact with said plaque.

2. Use of a biocompatible solvent as described in claim 2 where the said biocompatible solvent means is sufficiently insoluble in blood to allow discrete droplets of the said biocompatible solvent to travel through the cardiovascular system, from the entry point to the arterial plaque being targeted, without having more than 99% of the solvent dissolve into the blood or be solubilized by blood proteins.

3. Use of a biocompatible solvent as described in claim 1 where the said biocompatible solvent means has Hansen Solubility Parameters which are sufficiently well matched to cholesterol to allow some dissolution of the arterial plaque and where the Relative Energy Difference (RED value) as defined by Hansen solubility theory between the said biocompatible solvent and cholesterol is 1.3 or less.

4. Use of a biocompatible solvent as described in claim 1 where the said biocompatible solvent means has Hansen Solubility Parameters which are sufficiently well matched to cholesterol to allow some dissolution the of arterial plaque and where the Relative Energy Difference (RED value) as defined by Hansen solubility theory between the said biocompatible solvent and cholesterol is 1.0 or less.

5. Use of a biocompatible solvent as described in claim 1 where the said biocompatible solvent means has Hansen Solubility Parameters which are sufficiently well matched to cholesterol to allow some dissolution of the arterial plaque and where the Relative Energy Difference (RED value) as defined by Hansen solubility theory between the said biocompatible solvent and cholesterol is 0.8 or less.

6. Use of a biocompatible solvent as described in claim 3 where the biocompatible solvent means comprises of an organic compounds from the carboxylic acid family or organic molecules.

7. Use of a biocompatible solvent as described in claim 1 where a pure solution of the biocompatible solvent has been shown to be able to dissolve a mass fraction of cholesterol of greater than 1% at 37° C.

8. Use of a biocompatible solvent as described in claim 1 where a pure solution of the biocompatible solvent has been shown to be able to dissolve a mass fraction of cholesterol of greater than 2% at 37° C.

9. Use of a biocompatible solvent as described in claim 1 where a pure solution of the biocompatible solvent has been shown to be able to dissolve a mass fraction of cholesterol of greater than 6% at room temperature at 37° C.

10. Use of a biocompatible solvent as described in claim 1 which is further characterized by:
a) Use of a precipitation and filtration means 300 to filter the patient's blood and remove undissolved droplets of the said biocompatible solvent means from the patient's blood stream.

11. Use of a biocompatible solvent as described in claim 1 which is further characterized by:
a) Administering the said biocompatible solvent at a rate which exceeds the rate at which the patient's albumin and blood proteins are able to solubilize the said biocompatible solvent to ensure that a portion of the said biocompatible solvent travels within the cardiovascular system as undissolved (one word, I checked) droplets.
b) Use of a precipitation and filtration means 300 to filter the patient's blood and remove the said undissolved droplets of the said biocompatible solvent means from the patient's blood stream.

11. Use of a biocompatible solvent as described in claim 1 where the biocompatible solvent means comprises of one or more unsaturated fatty acids: α-Linolenic acid (ω-3), Stearidonic acid (ω-3), Eicosapentaenoic acid (ω-3), Docosahexaenoic acid (ω-3), Linoleic acid (ω-6), γ-Linolenic acid (ω-6), Dihomo-γ-linolenic acid (ω-6), Arachidonic acid (ω-6), Docosatetraenoic acid (ω-6), Palmitoleic acid (ω-7), Vaccenic acid (ω-7), Paullinic acid (ω-7), Oleic acid (ω-9), Elaidic acid (ω-9), Gondoic acid (ω-9), Errucic acid (ω-9), Nervonic acid (ω-9) or Mead acid (ω-9)

12. Use of a biocompatible solvent as described in claim 1 where the biocompatible solvent means comprises of one or more saturated fatty acids: Propionic acid (Propanoic acid), Butyric acid (Butanoic acid), Valerie acid (Pentanoic acid), Caproic acid (Hexanoic acid), enanthic acid (Heptanoic acid), Caprylic acid (Octanoioc acid), Pelargonic acid (Nonanoic acid), Capric acid (Decanoic acid), Undecylic acid (Undecanoic acid) or Lauric acid (Dodecanoic acid).

13. Use of a biocompatible solvent as described in claim 1 where the biocompatible solvent means comprises of a mixture of unsaturated fatty acids, saturated fatty acids and ethanol.

14. A system for treating or preventing one or more of heart disease, stroke, or atherosclerosis characterized by:
a) Means for Injecting a Biocompatible Solvent into the patients cardiovascular system or artery at a predetermined flow rate.
b) A biocompatible solvent means which has solubility parameters which are well matched to one of cholesterol or cholesterol esters and can be administered intravenously.

15. A system according to claim 14 further characterized by:
a) An pump means for controlling the flow rate of biocompatible solvent into the patient's cardiovascular system.
b) A filter means for removing air bubbles from the solvent being delivered to the patient
c) In input means such as a catheter for delivering the biocompatible solvent to a location in an artery which is slightly upstream of the plaque being treated.

16. A system according to claim 14 further characterized by:
a) A filtration and precipitation means for removing undissolved droplets of the said biocompatible solvent means from the patient's blood stream.

17. A system according to claim 16 where the filtration and precipitation means is further characterized by:
a) An input means for receiving blood from the patient
b) A pump means for drawing blood from the patient into the filtration and precipitation means
c) A filtration unit to remove undissolved droplets of the said biocompatible solvent means from the patient.
d) An outlet means for returning processed blood to the patient with a reduced quantity of undissolved biocompatible solvent.

18. A system according to claim 17 where the biocompatible solvent is filtered from the blood by leveraging the lower density of the said biocompatible solvent compared to that of blood.

19. A system according to claim 17 where the biocompatible solvent is filtered from the blood based on its hydrophobic characteristics.

20. A system according to claim 17 where the filtration and precipitation unit cools the blood to cause the biocompatible solvent means to solidify to facilitate separation of the said biocompatible solvent from the patient's blood.

21. A method for treating or preventing atherosclerosis, heart disease or stroke which comprises of:
a) Injecting a biocompatible solvent means into a patient's cardiovascular system to dissolve one of cholesterol, cholesterol esters or other fatty compounds from the arterial plaque in a patient's arteries

22. A method according to claim 21 where:
